(19) United States
(12) Patent Application Publication
(10) Pub. No.: US 2012/0058200 A1
(43) Pub. Date: Mar. 8, 2012

(54) PROCESS FOR THE PREPARATION OF LANTHANUM CARBONATE DIHYDRATE

(75) Inventors: Pulla Reddy Muddasani, Andhra Pradesh (IN); Sambasiva Rao Talasila, Andhra Pradesh (IN); Bhujanga Roa Adibhatla Kali Satya, Andhra Pradesh (IN); Venkata Reddy Satti, Andhra Pradesh (IN); Anil Kumar Lingamaneni, Andhra Pradesh (IN); Venkaiah Chowdary Nannapaneni, Andhra Pradesh (IN)

(73) Assignee: Natco Pharma Limited, Banjara Hills, Hyderabad (IN)

(21) Appl. No.: 13/319,050

(22) PCT Filed: May 15, 2009
(86) PCT No.: PCT/IN2009/000288

§ 371 Date: Nov. 4, 2011

(51) Int. Cl. A61K 33/24; A61P 3/12; C01F 17/00

(52) U.S. Cl. 424/617; 423/263

(57) ABSTRACT

Present invention relates to a process for the preparation and pharmaceutical usage of dihydrate form of lanthanum carbonate. Present process produces lanthanum carbonate dihydrate (La2(CO3)3.2H2O) free of lanthanum hydroxycarbonate and is stable at ambient condition. Lanthanum carbonate dihydrate exhibit improved performance over standard lanthanum carbonate tetrahydrate in phosphate binding studies. Lanthanum carbonate dihydrate is useful for the treatment of hyperphosphataemia in patients with renal failure.

### FIELD OF INVENTION

Present invention relates to a process for the preparation and pharmaceutical usage of dihydrate form of lanthanum carbonate. Selected Lanthanum carbonate hydrates are used to treat hyperphosphataemia in patients with renal failure. They are administered into the gastrointestinal tract. Shire Pharmaceuticals, under exclusive license from AnorMED, has developed and launched lanthanum carbonate (Fosrenol, formerly Foznol), a phosphate-binding lanthanum salt, for the oral treatment of hyperphosphataemia in dialysis patients. Lanthanum carbonate dihydrate has the formula given below.

La₂(CO₃)₃.xH₂O

Wherein x=2.0±0.2

### BACKGROUND OF INVENTION

Selected Lanthanum carbonate hydrates of formula La₂(CO₃)₃.xH₂O where x has a value from 3-6 are reported in the U.S. Pat. No. 5,968,976 by AnorMED (Canada) for the treatment of hyperphosphataemia by administration into the gastrointestinal tract. A process for the preparation of lanthanum carbonate tetrahydrate was also disclosed in this patent. Phosphate binding studies in this patent showed that samples of lanthanum carbonate with 3.8 to 4.4 moles of hydration are quicker in phosphate removal.

Chemical abstract search shows that the lanthanum carbonate is reported for the first time in 1923 (Z. Anaorg. Allgem. Chem., 131, 275-86, 1923). In the abstract no information regarding its preparation is disclosed. Crystal structure of lanthanum carbonate is reported in Geol. Nauk, #4, 157-95, 1954. No information regarding the preparation or degree of hydration is mentioned in the relevant abstract of this reference.

A process for the preparation of lanthanum carbonate is given in J. Am. Chem. Soc., 72, 3306, 1950. It is mentioned that pure crystalline rare earth compounds are difficult to prepare by the two commonly used methods, namely the precipitation of the compounds by alkali carbonates or bicarbonates from rare earth salt solutions or the conversion, in aqueous suspension, of rare earth hydroxides to carbonates by carbon dioxide. To solve these problems rare earth trichloroacetates were taken in water medium and heated to get pure carbonates. Here, the by-products are water and chloroform.

2La(C₂Cl₃O₂)₃+3H₂O→3CO₂+6CHCl₃+La₂(CO₃)₃

A process for the preparation of lanthanum carbonate octahydrate is disclosed in Izv. Akad. Nauk. SSSR, Neorgan. Materialy, 1(7), 1166-70 (1965). According to this process lanthanum nitrate is reacted with ammonium carbonate and the product isolated as octahydrate.

A process for the preparation of rare earth carbonates is discussed in J. Inorg. Nucl. Chem., 27, 1489-1493, 1965. According to this process water solutions of rare earth chlorides are reacted with ammonium trichloroacetate (prepared from ammonia and trichloroacetic acid) to get rare earth carbonates. Lanthanum carbonate prepared according to this process was isolated as octahydrate and its IR spectrum, thermogravimetric curve were given. Product was obtained in 50% yield.

Main drawback in this process is the usage of costly trifluoroacetic acid and low yield.

A process for the preparation of lanthanum carbonate is disclosed in U.S. Pat. No. 5,968,976. According to this reference, lanthanum oxide is converted to lanthanum nitrate using nitric acid. The resultant aqueous solution was reacted with sodium carbonate to get lanthanum carbonate. Alternatively, lanthanum oxide was reacted with hydrochloric acid to get lanthanum chloride. The resultant aqueous solution was reacted with sodium carbonate to get lanthanum carbonate.

Main drawback in this process is the usage of inorganic base, sodium carbonate. Removal of sodium salts from lanthanum carbonate is difficult. Also, lanthanum carbonate formed in the reaction is slimy in nature and filtration/washing of sodium nitrate is tedious.

Keeping in view of the difficulties in commercialization of the above-mentioned processes for the preparation of lanthanum carbonate, we aimed to develop a simple and cost effective process for commercial production of highly pure lanthanum carbonate.

### SUMMARY OF PRESENT INVENTION

Lanthanum carbonate hydrate is prone to decarboxylation under certain stressful conditions such as high temperature and humidity. The decarboxylation product is lanthanum hydroxycarbonate. Lanthanum hydroxycarbonate is known to exist in two polymorphic forms. An assay method for the quantification of lanthanum hydroxycarbonate in lanthanum carbonate hydrate by powder x-ray analysis is disclosed in EP1852695.

In the preparation of lanthanum carbonate disclosed in U.S. Pat. No. 5,968,976, lanthanum carbonate octahydrate is produced by reacting lanthanum chloride or nitrate with sodium carbonate in water medium. The resultant octahydrate is dried carefully at 80° C. for various durations of time to get the tetrahydrate derivative of lanthanum carbonate. Under these conditions, formation of lanthanum hydroxycarbonate is unavoidable.

We observed that lanthanum carbonate dihydrate exhibit improved performance over standard lanthanum carbonate tetrahydrate in phosphate binding studies.

One aspect of the present invention is the use of lanthanum carbonate dihydrate for the preparation of medicament for the treatment of hyperphasphataemia by administration into the gastrointestinal tract.

The present invention also provides a pharmaceutical composition comprising lanthanum carbonate dihydrate in admixture or association with a pharmaceutically acceptable diluent or a carrier, in a form suitable for administration into the gastrointestinal tract for the treatment of hyperphosphataemia.

We have now observed that lanthanum carbonate hydrate can be prepared by reacting readily available lanthanum chloride with an organic base such as ammonium bicarbonate to get free-flowing and fine crystalline lanthanum carbonate hydrate. Lanthanum carbonate hydrate can be easily isolated from the reaction mass by simple filtration and washing with minimum amount of water to remove the by-product, ammonium chloride. Also, we have now invented that a selected dihydrate of lanthanum carbonate can be obtained readily by drying under azeotropic conditions using a hydrocarbon solvent. Process for the preparation of dihydrate is robust without requiring any special/controlled drying condition as mentioned in U.S. Pat. No. 5,968,976 for similar hydrates.

According to one aspect, the present invention provides a process for the preparation of lanthanum carbonate dihydrate free of lanthanum hydroxycarbonate impurity, which comprises:
(i) Reacting lanthanum chloride hydrate with ammonium bicarbonate at 20-60° C. in water medium
(ii) Filtering the resultant lanthanum carbonate hydrate
(iii) Washing the wet lanthanum carbonate hydrate with water to get rid of the chlorides
(iv) Partial drying of lanthanum carbonate hydrate at 60-65° C.
(v) Suspending the partially dried lanthanum carbonate hydrate in a hydrocarbon solvent
(vi) Refluxing the medium under azeotropic conditions in the presence/absence of vacuum to get lanthanum carbonate dihydrate
(vii) Filtering and drying of the resultant lanthanum carbonate dihydrate at 60-65° C.

The reaction between lanthanum chloride and ammonium bicarbonate is given in the following equation:

2LaCl₃+6NH₄(HCO₃)→La₂(CO₃)₃+6NH₄Cl+3CO₂+3H₂O

Amount of water used in step (i) is selected from 30-70 volumes to the weight of lanthanum chloride, preferably 50-70 volumes. Preferred temperature of reaction in step (i) is 20-40° C., more preferably 25-35° C. Hydrocarbon solvent used in step (v) and (vi) is selected from hexane, heptane, cyclohexane, toluene, xylene, etc., preferably toluene.

Lanthanum carbonate hydrate produced according to the present invention is free flowing in nature and filtration was very fast compared to lanthanum carbonate hydrate produced according to U.S. Pat. No. 5,968,976 process. Also, lanthanum carbonate hydrate produced according to U.S. Pat. No. 5,968,976 is slimy in nature. Lanthanum carbonate dihydrate produced according to the present invention is free of lanthanum hydroxycarbonate impurity. Whereas lanthanum carbonate produced according to U.S. Pat. No. 5,968,976 is always contaminated with about 0.5-1.0% of this impurity. Lanthanum carbonate dihydrate is found to be stable at room temperature.

### BRIEF DESCRIPTION OF THE DRAWINGS

FIG. 1 Powder XRD of lanthanum carbonate dihydrate.

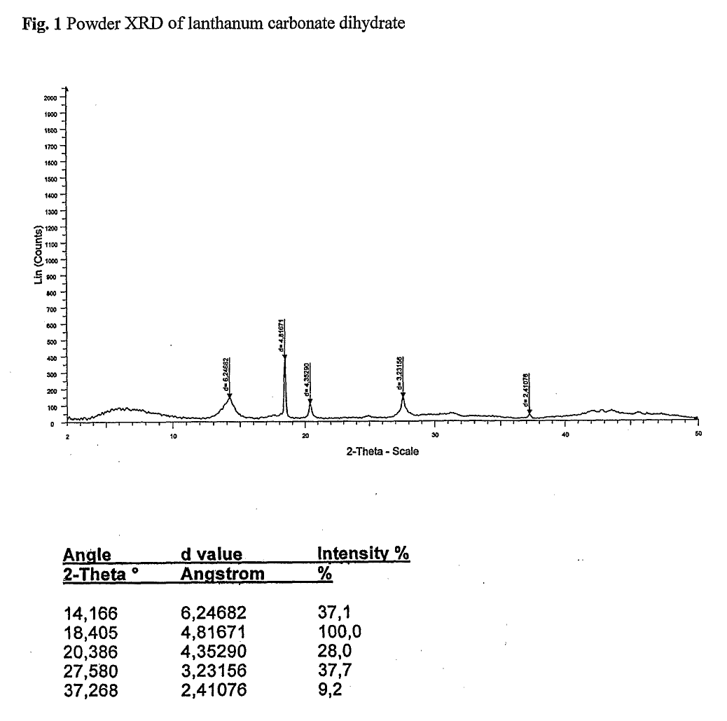

FIG. 2 IR (KBr) of lanthanum carbonate dihydrate

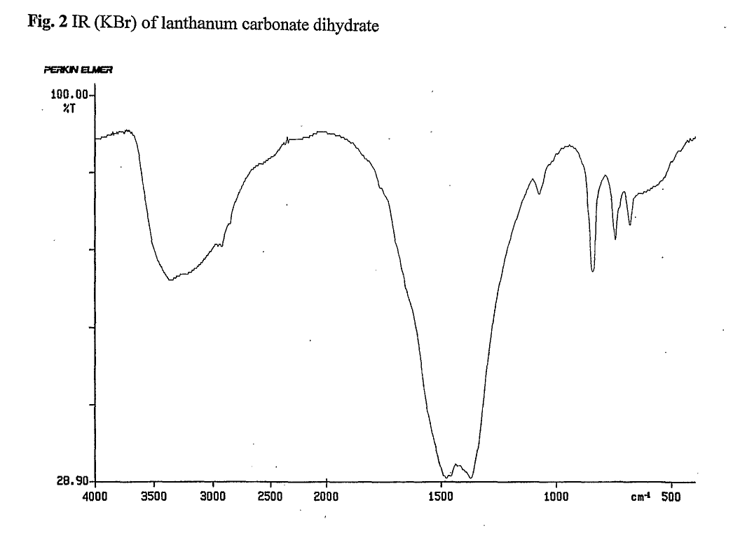

FIG. 3 TGA curve of lanthanum carbonate dihydrate

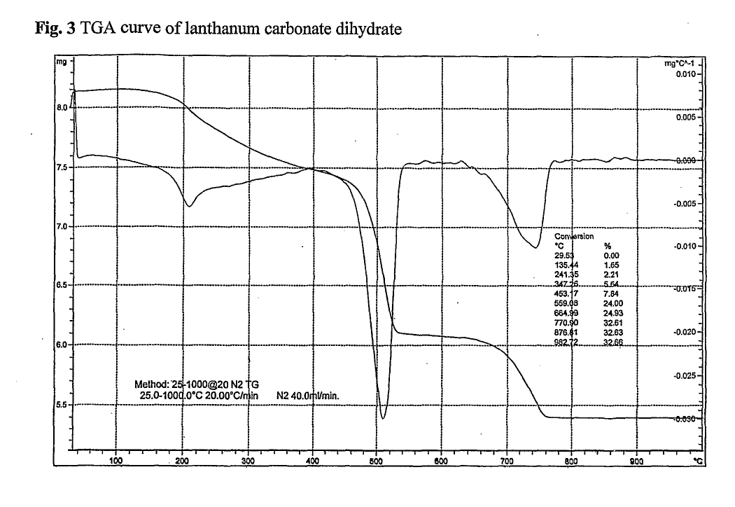

FIG. 4 Overlay of powder XRD of lanthanum carbonate with various percentages of lanthanum hydroxycarbonate Form I impurity with reference to a peak at 24.4 2 theta value

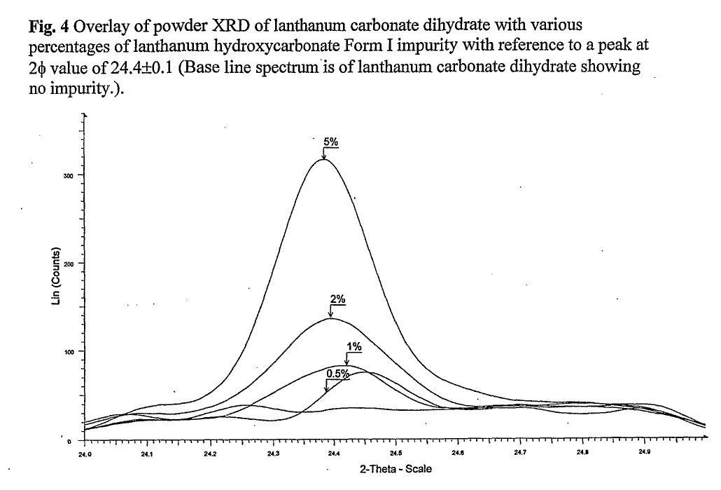

FIG. 5 Overlay of powder XRD of lanthanum carbonate with various percentages of lanthanum hydroxycarbonate Form II impurity with reference to a peak at 26.1 2-theta value

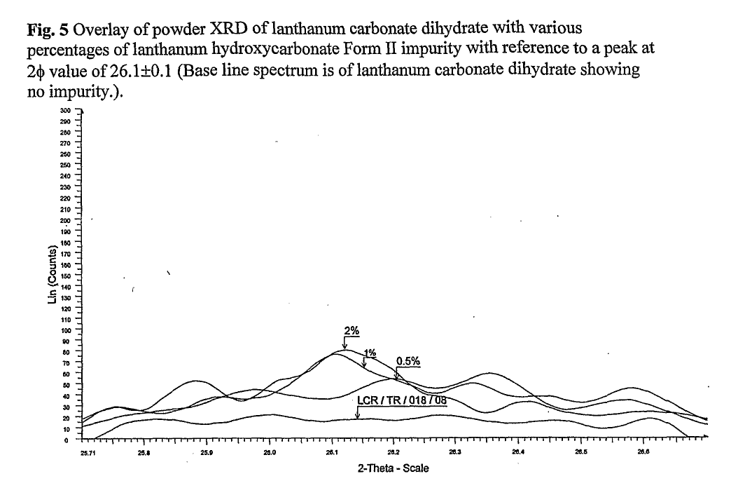

FIG. 6 Powder XRD of lanthanum hydroxycarbonate (Form I)

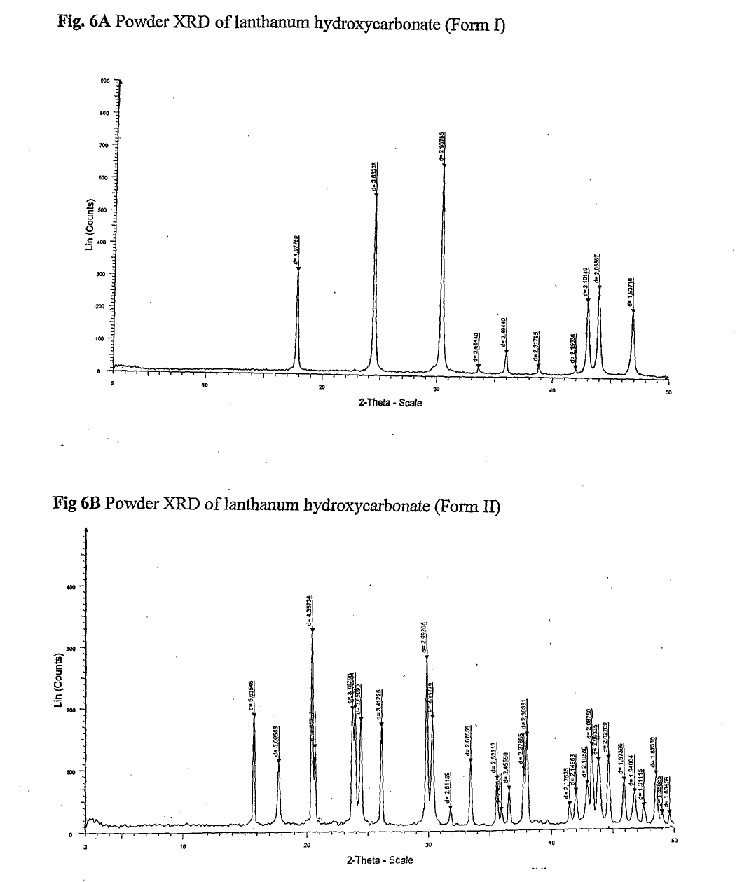

FIG. 7 Phosphate binding capability of lanthanum carbonate hydrates

The details of the invention are given in the Examples given below which are provided to illustrate the invention only and therefore should not be construed to limit the scope of the present invention.

### EXAMPLES

### Example 1

### Preparation of Lanthanum Carbonate Dihydrate

Into a 10 L, three-necked RB flask was charged 7 L of demineralized water. Lanthanum chloride heptahydrate (100 g) was charged into the flask and stirred for 30 min at 25-30° C. The solution was filtered through Bucher funnel and flask under vacuum to get a particle-free solution. Filtrate was transferred into a 10 L, three-necked RB flask.

Ammonium bicarbonate (130 g) was charged into a 2 L, three-necked RB flask and 700 ml of demineralized water was added. The resultant solution was filtered using a funnel and flask to make it particle-free. The filtrate was taken into an addition funnel and added slowly in 3-4 hours into lanthanum chloride solution at 25-30° C. After the completion of addition reaction, the mass was maintained for 1 hour at 25-30° C. The reaction mass was filtered through Buckner funnel and flask under vacuum. The wet cake was washed with 200 ml of demineralized water. The wet material was transferred into a 1 L, three-necked RB flask, 500 ml of demineralized water was added and stirred for 15 min. The mass was filtered though Buckner funnel and flask under vacuum. The chloride content in wet lanthanum carbonate hydrate was checked. The same washing procedure was repeated one more time, if the chloride content is above 500 ppm. The wet material was transferred into a petridish and dried in an oven at 60-65° C. for 4-6 hours.

The above lanthanum carbonate hydrate (69 g) was charged into a 1 L, four-necked RB flask. Particle free toluene (400 ml) was charged into the flask and the reaction mass was heated to reflux. Water was collected azeotropically using a Dean-Stark apparatus. When the water collection stopped (nearly 3-4 hours) the reaction mass was cooled to 30-35° C. The mass was filtered through Buchner funnel and flask under vacuum and finally washed with 100 ml of filtered toluene. The wet material was dried in an oven at 60-65° C. for 4-6 h to get lanthanum carbonate dihydrate (60 g) as white crystalline solid.

### Example 2

### Preparation of Lanthanum Carbonate Tablets

The lanthanum carbonate tablets were prepared using the above lanthanum carbonate dihydrate.

TABLE A

| | 1000 mg | | 750 mg | | 500 mg | | |
|---|---|---|---|---|---|---|---|
| Ingredient | Tablet | | Tablet | | Tablet | | Function |
| Lanthanum | 1777.50 | mg | 1333.12 | mg | 888.75 | mg | Active |
| carbonate | | | | | | | ingredient |
| dihydrate | | | | | | | |
| Dextrates | 2102.50 | mg | 1576.88 | mg | 1051.25 | mg | Diluent |
| (Hydrated) | | | | | | | |
| USP/NF | | | | | | | |
| Talc USP | 30.00 | mg | 22.50 | mg | 15.00 | mg | Glidant |
| Colloidal silicon | 30.00 | mg | 22.50 | mg | 15.00 | mg | Glidant |
| dioxide USP/NF | | | | | | | |
| Magnesium | 60.00 | mg | 45.00 | mg | 30.00 | mg | Lubri- |
| stearate USP/NF | | | | | | | cating |
| | | | | | | | agent |

| | | | | |
|---|---|---|---|---|
| Water | Qs | Qs | Qs | Vehicle |

| | | | | | | |
|---|---|---|---|---|---|---|
| Total | 4000 | mg | 3000 | mg | 2000 | mg |

TABLE B

| | 1000 mg | | 750 mg | | 500 mg | | |
|---|---|---|---|---|---|---|---|
| Ingredient | Tablet | | Tablet | | Tablet | | Function |
| Lanthanum | 1777.50 | mg | 1333.13 | mg | 888.75 | mg | Active |
| carbonate | | | | | | | ingredient |
| Dihydrate | | | | | | | |
| Dextrates | 821.70 | mg | 616.27 | mg | 410.85 | mg | Diluent |
| (Hydrated) | | | | | | | |
| USP/NF | | | | | | | |
| Dextrates | 105.80 | mg | 79.35 | mg | 52.90 | mg | Binder |
| (Hydrated) | | | | | | | |
| USP/NF | | | | | | | |
| Talc USP | 30.00 | mg | 22.50 | mg | 15.00 | mg | Glidant |
| Colloidal silicon | 25.00 | mg | 18.75 | mg | 12.50 | mg | Glidant |
| dioxide USP/NF | | | | | | | |
| Magnesium | 40.00 | mg | 30.00 | mg | 20.00 | mg | Lubri- |
| stearate USP/NF | | | | | | | cating |
| | | | | | | | agent |

| | | | | |
|---|---|---|---|---|
| Water | Qs | Qs | Qs | Vehicle |

| | | | | | | |
|---|---|---|---|---|---|---|
| Total | 2800 | mg | 2100 | mg | 1400 | mg |

### Example 3

Phosphate Binding Studies of Various Lanthanum Carbonate Hydrates

To study the phosphate binding activity of lanthanum carbonate dihdyrate, other hydrates of lanthanum carbonate (monohydrate, tetrahydrate and hexahydrate) were prepared.
a) A stock solution of standard phosphate was prepared by dissolving 13.75 g of Na₂HPO₄ in 1 L deionised water after adjusting the pH to 3.0 with conc. Hl.
b) Above stock solution (5 ml) was diluted with 90 ml water adjusted the pH to 3.0 with conc. HCl and made up to 100 ml with water.
c) A two-fold molar excess of Lanthanum carbonate hydrate over phosphate was weighed accurately according to the molecular weight and added to solution b).
d) Sampling was carried out at different time intervals 1, 3, 5, 7, and 10 min. The results are shown in Table C below.
   The results show that:
   1. Phosphate binding is always relatively very fast with the dihydrate at all points.
   2. Peak phosphate binding exceeding 99% is achieved with the dihydrate in about 7 times whereas only 81% is achieved with the tetrahydrate.

TABLE C
Phosphate binding studies with various hydrates of lanthanum carbonate

| Time | % Phosphate removed by | | | |
|---|---|---|---|---|
| (Minutes) | Monohydrate | Dihydrate | Tetrahydrate | Hexahydrate |
| 1 | 82.73 | 94.00 | 64.05 | 57.10 |
| 3 | 90.05 | 93.00 | 73.64 | 60.10 |
| 5 | 87.10 | 93.00 | 87.20 | 79.73 |
| 7 | 91.72 | 99.45 | 81.21 | 76.12 |
| 10 | 98.30 | 99.17 | 82.80 | 81.15 |

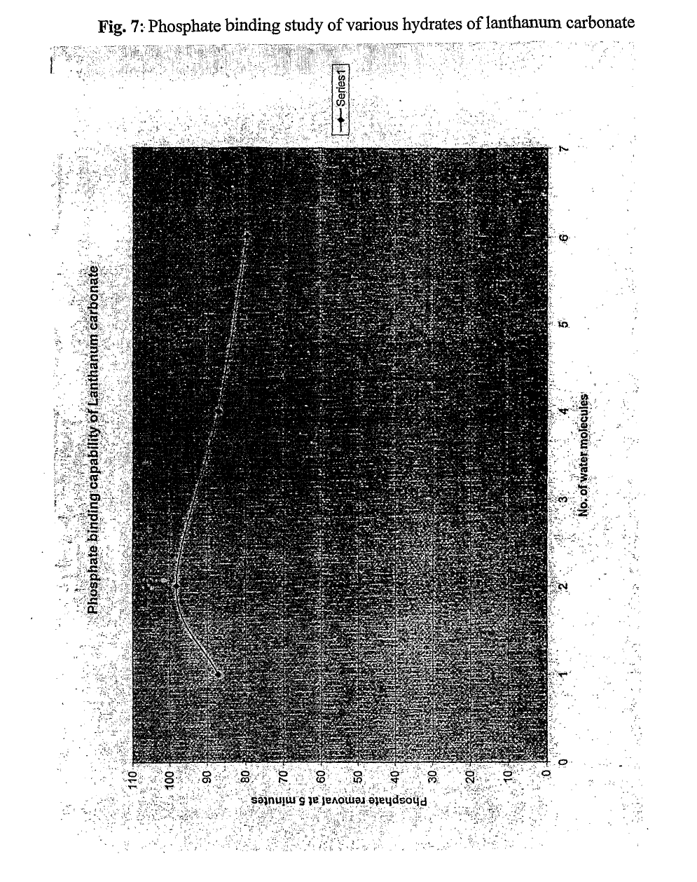

The above results are also plotted and shown in FIG. 7.

### Advantages of Present Invention

1. The present invention provides a process for the preparation of lanthanum carbonate dihydrate free of lanthanum hydroxycarbonate impurity.
2. Lanthanum carbonate dihydrate produced according to the process of present invention exhibit improved performance over standard lanthanum carbonate tetrahydrate in phosphate binding studies.
3. Lanthanum carbonate dihydrate is useful for the treatment of hyperphosphataemia in patients with renal failure.

1. A process for the preparation of stable lanthanum carbonate of the formula
La₂(CO₃)₃.xH₂O
wherein x=2.0±0.2
which comprises:
(i) reacting aqueous lanthanum chloride hydrate with aqueous ammonium bicarbonate at 20-60° C.
(ii) isolating the resultant lanthanum carbonate hydrate by filtration
(iii) washing the wet lanthanum carbonate hydrate with water to get rid of the chlorides
(iv) partial drying of lanthanum carbonate hydrate at 60-65° C.
(v) suspending the partially dried lanthanum carbonate hydrate in a hydrocarbon solvent
(vi) refluxing the medium under azeotropic conditions in the presence/absence of vacuum to get lanthanum carbonate dihydrate
(vii) filtering and drying of the resultant lanthanum carbonate dihydrate at 60-65° C.

2. The process according to claim 1 wherein the amount of water used in step (i) is 30-70 volumes to the weight of lanthanum chloride, preferably 50-70 volumes.

3. The process according to claim 1 wherein the temperature of reaction in step (i) is 20-40° C., preferably 25-35° C.

4. The process according to claim 1 wherein the amount of water present in lanthanum carbonate after partial drying is 15-20% w/w.

5. The process according to claim 1 wherein the hydrocarbon solvent used in step (v) and (vi) is selected from hexane, heptane, cyclohexane, toluene and xylene, and preferably is toluene.

6. The process according to claim 1 wherein the amount of water present in the lanthanum carbonate dihydrate product is 7.29±0.70% w/w.

7. (canceled)

8. A stable lanthanum carbonate hydrate of formula La₂(CO₃)₃.xH₂O, x=2.0±0.2, having a peak phosphate binding capacity of more than 99%.

9. A method as defined in claim 11 wherein the lanthanum carbonate dihydrate has a peak phosphate binding capacity of more than 99%.

10. A pharmaceutical composition comprising a lanthanum carbonate as defined in claim 8.

11. A method of treating hyperphosphataemia comprising administering to a patient a lanthanum carbonate dihydrate of the formula
La₂(CO₃)₃.xH₂O
wherein x=2.0±0.2.
